(19) United States
(12) Patent Application Publication
(10) Pub. No.: US 2007/0031397 A1
(43) Pub. Date: Feb. 8, 2007

(54) Compositions and methods for enhancing axon regeneration

(76) Inventors: Ronald L. Schnaar, Columbia, MD (US); Lynda J.S. Yang, Frederick, MD (US); Lawrence P. Schramm, Baltimore, MD (US)

Correspondence Address: EDWARDS ANGELL PALMER & DODGE LLP, P.O. BOX 55874, BOSTON, MA, 02205, US

(21) Appl. No.: 11/442,676

(22) Filed: May 25, 2006

Related U.S. Application Data
(60) Provisional application No. 60/684,340, filed on May 25, 2005.

(51) Int. Cl. A61K 38/47

(52) U.S. Cl. 424/94.61

(57) ABSTRACT

As described below, the present invention generally features compositions and methods for the treatment of CNS disease or injury. In particular, the invention provides methods and compositions for enhancing axonal outgrowth in a subject. In one embodiment, the invention enhances the success of CNS restorative surgery.

### CROSS-REFERENCE TO RELATED APPLICATION

This application claims the benefit of U.S. Provisional Application No.: 60/684,340, which was filed on May 25, 2005 the entire disclosure of which is hereby incorporated in its entirety.

### STATEMENT OF RIGHTS TO INVENTIONS MADE UNDER FEDERALLY SPONSORED RESEARCH

This work was supported by the following grants from the National Institutes of Health, Grant Nos: 5R21NS046669 AND 2R01NS037096. The government may have certain rights in the invention.

### BACKGROUND OF THE INVENTION

Spinal cord injuries and other injuries involving the central nervous system (CNS) are frequently caused by trauma. Often spinal cord injuries result in lifelong disabilities from loss of motor and sensory functions. Recovery from such injuries is typically poor because the injured CNS is a highly inhibitory environment for axon regeneration that severely limits functional recovery. This is due, in part, to axon regeneration inhibitors, specific molecules that accumulate at injury sites. Axon regeneration inhibitors include myelin-associated glycoprotein (MAG), NogoA, and oligodendrocyte-myelin glycoprotein (OMgp) on residual myelin and chondroitin sulfate proteoglycans on astrocytes of the glial scar. Some of these axon regeneration inhibitors bind to complementary receptors on axon growth cones and signal them to halt outgrowth.

One type of spinal cord injury is brachial plexus (nerve root) avulsion. The brachial plexus is a network of nerves that conducts signals from the spine to the shoulder, arm, and hand. Brachial plexus avulsion is a severe type of damage to this network that occurs when one or more of the brachial plexus nerves is torn from the spine. This type of injury is characteristic of >70% of all traumatic brachial plexus injuries. Regaining sensorimotor function after such injury was once thought to be impossible. Recently, nerve transfer has been used to provide biceps function and shoulder stability. Implantation of avulsed spinal nerve roots or peripheral nerve grafts into the spinal cord to bridge the CNS to the peripheral nervous system (PNS) has led to functional reconnection in some patients, but functional improvement has been limited by the presence of axon regeneration inhibitors in the microsurgical environment. Methods for enhancing axon outgrowth are urgently required to treat spinal cord injuries, in general, and to enhance the success of peripheral nerve graft implantation into the CNS in the treatment of brachial plexus avulsion.

### SUMMARY OF THE INVENTION

As described below, the present invention generally features compositions and methods for the treatment of CNS disease or injury.

In one aspect, the invention generally provides a method for enhancing axonal outgrowth in a cell by contacting the cell or cell substrate with an agent having sialidase or sialic acid modifying activity thereby enhancing axonal outgrowth. In one embodiment, the agent having sialidase activity or sialic acid modifying activity modifies a sialic acid present on the cell or on the substrate. In one embodiment, the cell is a cell of the central nervous system, such as a neuron (e.g., a motor or sensory neuron), for example, a neuron having an axon present in a brachial plexus. In another embodiment, the method enhances outgrowth from the CNS into a peripheral nerve graft. In yet another embodiment, the agent is administered prior to, during, or following restorative CNS surgery (e.g., peripheral nerve graft or a reinsertion of avulsed nerve roots). In yet another embodiment, the agent having sialidase activity is administered in combination with an agent having chondroitinase ABC activity. The combination may be administered concurrently or within 5 days of administration of the agent having chondroitinase ABC activity. In one embodiment, the administration increases axonal outgrowth by at least 2-fold relative to an untreated control condition.

In another aspect, the invention provides a method of blocking an axonal regeneration inhibitor in a subject in need thereof, the method involving administering to the subject an effective amount of an agent having sialidase or sialic acid modifying activity, thereby blocking an axonal regeneration inhibitor in the subject.

In a related aspect, the invention provides a method of enhancing axonal outgrowth in a subject in need thereof, the method involving administering to the subject an effective amount of an agent having sialidase or sialic acid modifying activity thereby enhancing axonal outgrowth. In one embodiment, the subject has a central nervous system disease or injury selected from the group consisting of stroke, head trauma, spinal injury (e.g., avulsion of the brachial plexus), ischemia, hypoxia, neurodegenerative disease, multiple sclerosis, infectious disease, cancer, and autoimmune disease. In another embodiment, the agent having sialidase or sialic acid modifying activity is administered to the subject prior to, during, or after restorative surgery (e.g., peripheral nerve graft or a reinsertion of avulsed nerve roots). In yet another embodiment, the agent having sialidase or sialic acid modifying activity is administered directly to the central nervous system of the subject, for example by infusion into the spinal cord by an osmotic pump, indwelling catheter, or sustained-release biomaterial. In yet another embodiment, the method enhances axonal outgrowth from the subject's CNS into a peripheral nerve graft. In yet other embodiments, the sialidase is administered concurrently or within 5 days of the administration of chondroitinase ABC.

In yet another aspect, the invention provides a method of enhancing axonal outgrowth in a subject having a spinal injury (e.g., brachial plexus avulsion), the method involving administering to a subject having CNS restorative surgery an effective amount of an agent having sialidase or sialic acid modifying activity thereby enhancing axonal outgrowth. In one embodiment, the method enhances outgrowth from the CNS into a peripheral nerve graft. In another embodiment, the method blocks or modifies the activity of an axonal regeneration inhibitor.

In yet another aspect, the invention provides a method for identifying an agent that enhances axonal outgrowth, the method involving contacting a neuron in the presence of an axonal regeneration inhibitor with an agent having sialidase activity or sialic acid modifying activity; and comparing axonal outgrowth in the presence of the agent relative to a control condition, where an increase in axonal outgrowth in the presence of the agent thereby identifies the agent as enhancing axonal outgrowth.

In a related aspect the invention provides a method for identifying an agent that enhances axonal outgrowth. The method involves contacting an axonal regeneration inhibitor with an agent having sialidase activity or sialic acid modifying activity; and identifying a biochemical modification of the axonal regeneration inhibitor, where an agent that biochemically modifies the axonal regeneration inhibitor is identified as enhancing axonal outgrowth. In various embodiments of the above aspect, the invention further involves contacting a neuron in the presence of an axonal regeneration inhibitor with the agent; and comparing axonal outgrowth in the presence of the agent relative to a control condition, where an increase in axonal outgrowth in the presence of the agent identifies the agent as enhancing axonal outgrowth. In still other embodiments, the agent is a sialidase polypeptide fragment, variant or analog.

In yet another aspect, the invention provides a pharmaceutical composition for use in enhancing axonal outgrowth in a subject in need thereof, the composition containing an effective amount of an agent having sialidase activity or sialic acid modifying activity in a pharmaceutically acceptable excipient.

In a related aspect, the invention provides a pharmaceutical composition for use in enhancing axonal outgrowth in a subject in need thereof, the composition containing an effective amount of an agent having sialidase activity in a pharmaceutically acceptable excipient.

In a related aspect, the invention provides a pharmaceutical composition for use in enhancing axonal outgrowth in a subject in need thereof, the composition containing effective amounts of sialidase and chondroitinase ABC in a pharmaceutically acceptable excipient. In another aspect, the invention provides a therapeutic delivery device containing an agent having sialidase activity or sialic acid modifying activity, where the device locally releases the agent into the CNS for the treatment of a CNS disease or injury. In one embodiment, the device further contains an agent having chondroitinase ABC activity. In another embodiment, the device is an osmotic pump, indwelling catheter, or sustained-release biomaterial.

In various embodiments of the above aspects, the agent is sialidase. In still other embodiments of the above aspects, the agent having sialidase activity modifies sialoglycoconjugates, cleaves terminal sialic acids, or modifies gangliosides, sialoglycoproteins, or polysialic acid present on the cell, on the substrate, or in the cellular environment. In still other embodiments, the modified gangliosides are GD1a and GT1b. In other embodiments of the above aspects, at least about 0.1, 0.2, 0.3, 0.5, 0.75, 1, 2, 3, 4, or 5 U/ml of chondroitinase ABC is administered. In still other embodiments, between about 0.1, 0.2, 0.3, 0.5, 0.75, 1, 2, 3, 4, or 5 U/ml of sialidase is administered. In still other embodiments, the administration increases axonal outgrowth by at least 2-fold relative to an untreated control. In still other embodiments, a combination of agents having sialidase and chondroitinase ABC activity is administered. When administered in combination, the agents (e.g., sialidase and chondroitinase ABC) are administered concurrently or within at least 1, 2, 3, 4, 5, 7 or 10 days.

Other features and advantages of the invention will be apparent from the detailed description, and from the claims.

### Definitions

By “agent” is meant a polypeptide, peptide, nucleic acid molecule, small molecule, or mimetic.

By “analog” is meant an agent having structural or functional homology to a reference agent.

By “axonal regeneration inhibitor” is meant any agent that slows or decreases axonal outgrowth. In vitro and in vivo assays for axonal outgrowth are known in the art and are described herein.

By “blocking an axonal regeneration inhibitor” is meant the biochemical modification or other action that tends to decrease the efficacy of an axonal regeneration inhibitor. An agent that “blocks” an axonal regeneration inhibitor increases axonal outgrowth in an in vitro or in vivo assay where a neuron is contacted with an axon regeneration inhibitor in the presence or absence of the blocking agent (e.g., sialidase or chondroitinase ABC).

By “cell substrate” is meant the cellular or acellular material (e.g., extracellular matrix, polypeptides, peptides, or other molecular components) that is in contact with the cell.

By “cellular environment” is meant the area directly surrounding and in direct contact with neurons and their axons.

By “central nervous system” (CNS) is meant the brain or spinal cord, and cellular or molecular components thereof, including the extracellular materials and fluids.

By “central nervous system disease or injury” is meant any disease, disorder, or trauma that disrupts the normal function or connectivity of the brain or spinal cord.

By “chondroitinase ABC” is meant a chondroitinase ABC polypeptide or fragment thereof having at least 50% of the enzymatic activity of a wild-type chondroitinase enzyme. In other embodiments, the chondroitinase polypeptide is a fragment, variant, or analog of chondroitinase ABC having at least 65%, 75%, 85%, 95% of the activity of a wild-type enzyme. In other embodiments, the fragment, variant, or analog has increased activity (e.g., 2, 3, 5, or 10 times the activity of a naturally occurring enzyme. An exemplary chondroitinase ABC amino acid sequence is provided at NCBI Accession No. P59807 (Proteus vulgaris)

By “control” is meant a standard or reference condition.

By “disease” is meant any condition or disorder that damages or interferes with the normal function of a cell, tissue, or organ.

By “effective amount” is meant the amount of an agent required to ameliorate the symptoms of a disease relative to an untreated patient. The effective amount of an active therapeutic agent used to practice the present invention for the treatment of a CNS disease or injury varies depending upon the manner of administration, the age, body weight, and general health of the subject. Ultimately, the attending clinician will decide the appropriate amount and dosage regimen. Such amount is referred to as an “effective” amount.

By “enhancing axonal outgrowth” is meant increasing the number of axons or the distance of extension of axons relative to a control condition. Preferably the increase is by at least 2-fold, 2.5-fold, 3-fold or more.

By “fragment” is meant a portion of a polypeptide that has at least 50% of the biological activity of the polypeptide from which it is derived. This portion contains, preferably, at least 10%, 20%, 30%, 40%, 50%, 60%, 70%, 80%, or 90% of the entire length of the reference nucleic acid molecule or polypeptide. A fragment of a polypeptide or nucleic acid molecule may contain 10, 20, 30, 40, 50, 60, 70, 80, 90, or 100, 200, 300, 400, 500, 600, 700, 800, 900, or 1000 nucleotides or amino acids.

By “modifies” is meant alters. In the context of the invention, an agent that modifies a cell, substrate, or cellular environment produces a biochemical alteration in a component (e.g., polypeptide, nucleotide, or molecular component) of the cell, substrate, or cellular environment.

By “neuron” is meant any nerve cell derived from the nervous system of a mammal.

By “peripheral nerve graft” is meant any cellular or non-cellular material derived from the peripheral nervous system that is implanted into a heterologous environment. In one approach, the peripheral nerve graft generally comprises an acellular matrix that supports axonal extension.

By “restorative CNS surgery” is meant any procedure carried out on the central nervous system to enhance neurological function. An exemplary restorative CNS surgery is a peripheral nerve graft or a reinsertion of avulsed nerve roots.

By “sialidase” is meant a sialidase polypeptide or fragment thereof having at least 50% of the enzymatic activity of a wild-type sialidase enzyme. In other embodiments, the sialidase polypeptide is a fragment, variant, or analog of a naturally occurring sialidase that has at least 65%, 75%, 85%, 95% of the activity of the wild-type enzyme. In other embodiments, the fragment, variant, or analog has increased activity (e.g., 2, 3, 5, or 10 times the activity of a naturally occurring enzyme). Exemplary sialidases include, but are not limited to, amino acid sequences provided at NCBI Accession No. CAA44916 (Clostridium perfringens), AAA27546 (Vibrio cholerae), P29768 (Salmonella typhimurium LT2), BAB40435 (Erysipelothrix rhusiopathiae), AAC95494 (Trypanosoma rangeli), BAD66680 (Arthrobacter ureafaciens), CAA44166 (Actinomyces viscosus), BAB39152 (Mus musculus), BAB32z440 (Rattus norvegicus), CAA55356 (Homo sapiens), and CAB96131 (neuraminidase, Homo sapiens). The skilled artisan will appreciate that the term “sialidase” is used interchangeably with the term “neuraminidase” in the scientific literature.

By “sialic acid modifying activity” is meant any biochemical modification of sialic acid. Such modifications include additions to or deletion of a hydroxyl group, an N-acetyl group, or carboxylic acid. Exemplary modifications include, but are not limited to, addition of a hydroxyl group to the sialic acid N-acetyl group and addition of an acetyl group to a sialic acid hydroxyl group. Exemplary polypeptides having sialic acid modifying activity include, but are not limited to, N-acetylneuraminic acid hydroxylase (NCBI Accession No. Q8MJC8) and Sialic Acid O-Acetyltransferase (NCBI Accession No. Q8MJC8 and AAG43983).

By “subject” is meant a mammal, including, but not limited to, a human or non-human mammal, such as a bovine, equine, canine, ovine, or feline.

By “therapeutic delivery device” is meant any device that provides for the release of a therapeutic agent. Exemplary therapeutic delivery devices include osmotic pumps, indwelling catheters, and sustained-release biomaterials.

By “variant” is meant an agent having structural homology to a reference agent but varying from the reference in its biological activity. Variants provided by the invention include optimized amino acid and nucleic acid sequences that are selected using the methods described herein as having one or more desirable characteristics.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

### BRIEF DESCRIPTION OF THE DRAWINGS

FIGS. 1A and 1B are photographs of a rat model of brachial plexus nerve avulsion injury with peripheral nerve graft. FIG. 1A shows a surgical preparation prior to closure of the spinal cord in the region of C8 with the peroneal nerve graft extending from its insertion site in the ventrolateral aspect of the spinal cord toward its coaptation with the suprascapular nerve, which is not visible. A catheter extending from an osmotic pump is anchored, via a suture, to the dura just caudal to the graft insertion site. FIG. 1B shows a fixed preparation of the rat model. After perfusion-fixation of the rat, the spinal cord, peroneal nerve graft, and coapted suprascapular nerve were dissected, allowing visualization of the bridging graft.

FIGS. 2A-I are photomicrographs showing the in vivo efficacy of infused enzymes. Immunohistochemistry was carried out on horizontal sections from control animals (left panels A,B,D,F and H) and enzyme-infused animals (right panels C,E,G and I). Primary monoclonal antibodies were as follows: Panel A, none (control); panels B & C, anti-GT1b; panels D & E, anti-GM 1; panels F & G, anti-Thy- 1; and panels H & I, monoclonal antibody 2-B-6 against chondroitinase ABC lyase product. Enzyme treatments were sialidase (0.4 U/ml, panels C & E), phosphatidylinositol-specific phospholipase C (PI-PLC, 2 U/ml, panel G) and chondroitinase ABC (0.5 U/ml, panel I). Note the asymmetric decrease or increase in immunostaining on the lateral side of the cord, presumably due to enhanced enzyme activity near the tip of the infusion catheter, which was placed laterally. Scale bar=1 mm.

FIGS. 3A-3F are photomicrographs showing rat spinal neurons retrogradely labeled via a peroneal nerve graft. A retrograde tracer was used to label axons 4 weeks after implantation. The peroneal nerve graft was re-cut 7 mm distal to its spinal cord insertion site and sealed in a micro-reservoir of Fluoro-ruby dye. Horizontal sections of the spinal cord are shown in the area surrounding the peripheral nerve graft. The graft is visible as a roughly circular cross section containing labeled spinal axons, surrounded by retrogradely labeled spinal neurons. Red fluorescent images (indicating Fluoro-ruby retrograde staining) are presented as reverse grayscale for clarity. FIG. 3A shows control (saline) treated animals, which display some retrogradely labeled neurons. FIG. 3B shows sections taken from rats treated with PI-PLC (20 U/ml). These sections appear similar to those taken from control animals. In contrast, sections taken from rats treated with sialidase (0.38 U/ml, FIGS. 3C,D) and chondroitinase ABC (0.5 U/ml, FIGS. 3E,F) display significantly greater numbers of stained axons and spinal neurons. Most of the retrogradely labeled neurons are adjacent to the graft. Bar=200 μm.

FIG. 4 is a graph showing the quantification of retrogradely labeled spinal neurons in control and enzyme-treated animals. Average total retrograde-labeled neurons in spinal cord sections from animals treated with saline (Control, n=12), chondroitinase ABC (ChABC,n=11), PI-PLC (n=6 each concentration), and sialidase (n=6 each concentration) are shown (mean±SEM). Treatment with chondroitinase ABC (0.5 U/ml) and sialidase (0.38 U/ml) each resulted in significantly greater peripheral nerve graft innervation compared to the saline-treated control (*, p≦0.005 Student's T test).

FIGS. 5A, 5B, and 5C are spinal cord sections showing innervation of peripheral nerve grafts by remote neurons. Although most of the retrogradely labeled neurons are adjacent to the graft (see FIG. 3), it was not uncommon to find more distant labeled neurons near the central canal or on the contralateral side (FIGS. 5A, 5B, arrows; FIG. 5B insert). In some sections, the continuity of axons was traced from distant labeled spinal neurons to the graft (FIGS. 5A, 5B, color-enhanced). Some axons spanned multiple segments in the horizontal sections (FIG. 5C, arrowheads). Treatments were: FIG. 5A, 0.38 U/ml sialidase; FIG. 5B and FIG. 5C, 0.5 U/ml chondroitinase ABC. Bar=200 μm (insert bar=50 μm).

### DETAILED DESCRIPTION OF THE INVENTION

The invention generally features compositions and methods that are useful for treating central nervous system disease or injury. In particular, the invention provides methods for enhancing axonal outgrowth in a subject. The invention is based in part on the discovery that sialidase and chondroitinase ABC each enhances axonal outgrowth in vivo following spinal cord injury. Methods of the invention are particularly useful for treating traumatic injury of the CNS or spinal cord, and for enhancing the success of CNS restorative surgery, such as peripheral nerve graft implantation.

Axonal Regeneration in the CNS

The injured central nervous system (CNS) is a highly inhibitory environment for axon regeneration, severely limiting functional recovery following a traumatic injury. This is due, in part, to axon regeneration inhibitors, which are specific molecules that accumulate at injury sites. Axon regeneration inhibitors include myelin-associated glycoprotein (MAG), NogoA, and oligodendrocyte-myelin glycoprotein (OMgp) on residual myelin and chondroitin sulfate proteoglycans (CSPG's) on astrocytes of the glial scar. Some of these axon regeneration inhibitors bind to complementary receptors on axon growth cones and signal them to halt. Reducing the activity of axon regeneration inhibitors enhances axon outgrowth and recovery after CNS injury.

In contrast to the CNS environment, peripheral nerve sheathes support axon outgrowth, making peripheral-central nerve grafts an appealing therapeutic target for agents that block axonal regeneration inhibitors. Enhancement of CNS axon growth into peripheral nerve grafts is likely to translate into enhanced target innervation and function. One therapeutic application of peripheral nerve graft implantation into the CNS is in the treatment of brachial plexus avulsion. Upon nerve root avulsion, the microscopic environment of the severed axons within the CNS is highly inhibitory as evidenced by characteristic terminal retraction balls on axon pathways between the ventral horn and the pia mater¹.

Axonal Regeneration Inhibitors

Axon regeneration inhibitors and their axonal receptors are known in the art and are listed in Table 1.

TABLE 1
Axon regeneration inhibitors

| ARI | Source | Axonal receptor | Enzyme Modifier |
|---|---|---|---|
| NogoA | residual myelin | NgR family | PI-PLC |
| OMgp | residual myelin | NgR family | PI-PLC |
| MAG | residual myelin | NgR family | PI-PLC |
| MAG | residual myelin | sialoglycoconjugates | sialidase |
| CSPG | reactive astrocytes | unknown | chondroitinase ABC |

Compositions that target these axon regeneration inhibitors provide new molecular therapies to reduce axon regeneration inhibitor activity. The transmembrane myelin proteins NogoA and myelin-associated glycoprotein (MAG), and the glycosylphosphatidylinositol(GPI)-anchored myelin protein oligodendrocyte myelin glycoprotein (Omgp) are postulated to act by binding to a GPI-anchored family of receptors on axons, the Nogo receptors (NgR's)²⁻⁴. MAG is also a member of the Siglec family of sialic acid binding lectins ⁵, and has been proposed to inhibit axon regeneration by binding to axonal sialoglycoconjugates, including gangliosides GD1a and GT1b⁶⁻⁹. Although the axonal receptor for chondroitin sulfate proteoglycan (CSPG) is unknown, its glycosaminoglycan chains are required for inhibiting axon outgrowth¹⁰. These findings provide opportunities to block axon regeneration inhibitor function by infusing enzymes to injury sites.

Therapeutics that Block Axonal Regeneration Inhibitors

A number of enzymes are known to biochemically disrupt axonal regeneration inhibitors. Chondroitinase ABC, for example, digests the glycosaminoglycan chains of chondroitin sulfate proteoglycan¹⁰. Phosphatidylinositol-specific phospholipase C (PI-PLC) removes Nogo receptors from the axon surface and OMgp from myelin¹¹⁻¹². Sialidase destroys the glycan binding determinant of MAG^{6, 9, 13}. As reported in more detail below, sialidase and chondroitinase ABC each enhanced axonal outgrowth when administered in conjunction with a peripheral nerve graft in a rat model of brachial plexus avulsion.

Avulsion occurs in more than 70% of brachial plexus injuries¹⁴, and avulsion injury involving the ventral roots has a poor capacity for functional regeneration because of the physical separation of the axons and their nerve sheathes from their corresponding nerve cell bodies within the central nervous system¹. The mainstay of treatment is surgical and includes palliative surgery, such as nerve or muscle transfers, and restorative surgery, such as the implantation model used in the current study^{15, 16-19}. Two types of spinal cord implantation can restore connections between ventral horn neurons and their peripheral targets: reimplantation of the avulsed roots into the spinal cord and implantation of grafts between the spinal cord and distal nerve stumps or muscles. Reimplantation, however, is not an option if the avulsed nerve roots retract, which commonly occurs during the delay between injury and surgery. While grafts used in humans have ranged from artificial substrata to freeze-dried muscles, autologous nerve grafts offer the greatest viability¹⁶.

Reinnervation of muscle does not significantly restore function unless the regenerated axons establish appropriate contacts with the muscle in a timely fashion. The inherently slow rate of axon regeneration can limit clinical efficacy of treatments designed to restore function. Because graft implantation carries its own risks of spinal cord injury, surgeons who perform this procedure have called for optimizing functional outcomes using neurobiological strategies to improve regeneration^{15,16 20}. As reported herein, sialidase and chondroitinase ABC each enhance axon regeneration into peripheral nerve grafts implanted into the spinal cord, and provide two potential therapeutic targets to improve regeneration. Accordingly, the invention provides agents having sialidase and/or chondroitinase ABC activity that reduce the level or biological activity of an axonal regeneration inhibitor. Such agents enhance axonal outgrowth and are useful for the treatment of CNS disorders, such as spinal injury, where a restoration of sensorimotor connectivity is required.

Sialidases

Sialidases (also known as neuraminidases) are a family of glycohydrolytic enzymes that cleave sialic acid residues from the oligosaccharide components of glycoproteins and glycolipids, including sialo-oligosaccharides, gangliosides, or sialo-glycoproteins. Sialidases are found in a variety of organisms, including bacteria, viruses, protozoa, and vertebrates. Agents having sialidase activity are useful in the methods of the invention. Such agents include, but are not limited to, polypeptides having sialidase activity, biologically active fragments thereof, sialidase analogs and variants, as well as nucleic acid molecules encoding such agents. In general, any composition that modifies a sialoglycoconjugate, that catalyzes the hydrolysis of a terminal sialic acid linked to an oligosaccharide through an O-glycosidic bond, that enhances axonal outgrowth in an in vitro or in vivo assay, or that decreases or blocks the activity of an axonal regeneration inhibitor maybe used in the methods of the invention. Compositions having sialidase activity include bacterial sialidases from Clostridium perfringens, Vibrio cholerae, Arthrobacter ureafaciens, and Salmonella typhimurium. Mamalian sialidases are also useful in the methods of the invention and have been identified in a number of cellular organelles including the plasma membrane (Schengrund et al. (1976) J. Biol. Chem., 79:555), the lysosomes and the cytosol (Tulsiani et al., (1970) J. Biol. Chem., 245:1821). Other sialidases useful in the methods of the invention are described, for example, in U.S. Pat. Nos. 6,114,386 and 5,312,747.

Methods of assaying sialidase activity are known in the art and are described herein in Example 1. See, also U.S. Pat. No. 6,844,346. A sialidase polypeptide may be isolated from a cell or organism that endogenously expresses it or may be expressed as a recombinant polypeptide in a suitable expression system. Such methods are known in the art and are described, for example, in U.S. Pat. No. 6,436,687. In order to provide quantities of a sialidase polypeptide sufficient for therapeutic or other use, appropriate coding sequences may be introduced into host cells which will express the polypeptide. See, for example, Moustafa et al. (2004) “Sialic acid Recognition by Vibrio cholerae Neuraminidase”, J. Biol. Chem. 279: 40819-40826. Those of skill in the art will recognize that a variety of possible expression systems exist and are well-known, as are the methods for introducing a vector which encodes the polypeptide, and various means for isolating and purifying a polypeptide of interest. Examples of suitable expression systems, include but are not limited to bacterial host cell systems (e.g. Escherichia coli), and eukaryotic systems (e.g. yeast, mammalian cells, and insect cells). In one embodiment, a recombinant sialidase polypeptide or biologically active fragment thereof is introduced to a site of CNS disease or injury by local administration (e.g., by infusion). In another embodiment, a nucleic acid molecule encoding a sialidase polypeptide or biologically active fragment thereof is introduced directly into cells at a site of CNS disease or injury via genetic means, for example, by introducing into the desired cells a gene that encodes a sialidase polypeptide. Those of skill in the art will recognize that a variety of means exist for introducing nucleic acids into cells, including but not limited to the use of carrier molecules (e.g. vectors and lipids) as described below. The sialidase nucleic acid molecules and sialidase enzyme of the invention can be incorporated into pharmaceutical compositions suitable for administration. If desired, the sialidase polypeptide, biologically active fragment, or variant thereof is introduced in combination with another agent that blocks axonal regeneration inhibitors, such as chondroitinase ABC.

Chondroitinases

Chondroitinase ABC is one exemplary chondroitinase enzyme that degrades axonal regeneration inhibitors, including chondroitin sulfate proteoglycans. Chondroitinases are useful in combination with agents having sialic acid modifying activity for the enhancement of axonal outgrowth. Chondroitinase ABC may be isolated from the organisms or cells that produce them, or may be recombinantly expressed. In one embodiment, a chondroitinase ABC nucleic acid sequence is derived from Proteus vulgaris (See, Prabhakar et al., Biochem J.(2005) 386: 103-112) and a recombinant protein is generated for use in the methods of the invention. ]

The methods of the invention are broadly applicable to the treatment of CNS disease or injury. In this regard, the therapeutic methods described herein are useful for treatment of injury to the brain and spinal cord due to trauma, ischemia, hypoxia, neurodegenerative disease, infectious disease, cancer, autoimmune disease and metabolic disorder. Exemplary CNS diseases or injuries include stroke, head trauma, spinal injury, hypotension, arrested breathing, cardiac arrest, Reye's syndrome, cerebral thrombosis, embolism, cerebral hemorrhage, brain tumors, encephalomyelitis, hydroencephalitis, operative and postoperative brain injury, Alzheimer's disease, Huntington's disease, Creutzfeld-Jakob disease, Parkinson's disease, multiple sclerosis and amyotrophic lateral sclerosis. Thrombus, embolus, and systemic hypotension are the most common causes of cerebral ischemic episodes. Other causes of cerebral ischemia include hypertension, hypertensive cerebral vascular disease, rupture of an aneurysm, an angioma, blood dyscrasias, cardiac failure, cardiac arrest, cardiogenic shock, septic shock, head trauma, spinal cord trauma, seizure, bleeding from a tumor, or other blood loss. With respect to CNS trauma, trauma can involve a tissue insult such as an abrasion, incision, contusion, puncture, or compression. Such injuries can arise from traumatic contact of a foreign object with the head, neck, or vertebral column. Other forms of traumatic injury can arise from constriction or compression of the CNS tissue by an inappropriate accumulation of fluid (for example, a blockade or dysfunction of normal cerebrospinal fluid or vitreous humor fluid production, turnover, or volume regulation, or a subdural or intracranial hematoma or edema). Similarly, traumatic constriction or compression can arise from the presence of a mass of abnormal tissue, such as a metastatic or primary tumor.

The methods of the invention comprise administering a therapeutically effective amount of a pharmaceutical composition having sialidase activity or chondroitinase ABC activity or related compounds to a site where axonal outgrowth is required in a subject (e.g., a mammal, such as a human). In one embodiment the invention provides a method of treating a subject suffering from a spinal cord or central nervous system injury, or a related disease or disorder or symptom thereof that involves administering to the subject a therapeutic amount of an amount of a compound sufficient to treat the injury, disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof.

Treatment of CNS Disease or Injury

The results reported herein demonstrate that sialidase or chondroitinase ABC administration promotes axonal outgrowth in vivo. Accordingly, agents having sialidase activity can be provided alone or in combination with chondroitinase ABC to enhance axonal outgrowth or sensorimotor connectivity. Alternatively, nucleic acid molecules encoding such compositions are provided for expression in a cell at a site of CNS injury or disease where axonal outgrowth is desired.

Agents having sialic acid modifying activity are particularly useful in cellular environments within the CNS where the presence of axonal regeneration inhibitors limits axonal outgrowth. In one embodiment, sialidase or a combination of sialidase and chondroitinase is delivered locally to a site of CNS disease or injury where axonal regeneration is required. The agent is delivered in a form sufficient to increase, for example, axonal outgrowth or to restore neuronal connectivity, where such connectivity has been disrupted by disease or injury. Typically, neuronal connectivity is restored when a damaged neuron establishes a functional connection with a target neuron or muscle.

One therapeutic approach included in the invention involves administration of a recombinant therapeutic, such as a sialidase or a combination of sialidase and chondroitinase, a biologically active fragment, variant or analog thereof, either directly to the site of CNS injury or to an actual disease-affected tissue. In one embodiment, a therapeutic agent is locally administered to the site of injury or disease by local injection via a catheter or osmotic pump, by infusion or by delivery to cerebrospinal fluid in communication with the site. Alternatively, the agent is delivered systemically using any conventional recombinant protein administration technique. The dosage of the administered protein depends on a number of factors, including the size and health of the individual patient. For any particular subject, the specific dosage regimes should be adjusted over time according to the individual need and the professional judgment of the person administering or supervising the administration of the compositions. Generally, between 0.1 mg and 100 mg, is administered per day to an adult in any pharmaceutically acceptable formulation. For sialidase, dosages between 0.1, 0.2, 0.3, 0.38, 0.5, and 1.0 U/ml are used. For chondroitinase ABC dosages between 0.1, 0.2, 0.3, 0.4, 0.5, 1, 2, 3, 4, and 5.0 U/ml are used.

The expression or activity of a sialidase nucleic acid molecule or polypeptide is one therapeutic approach for preventing or ameliorating a CNS disease or injury where an increase in axonal outgrowth or a decrease in axonal regeneration inhibitor activity is desirable. In one embodiment, a nucleic acid molecule encoding sialidase is delivered to cells at a site of CNS disease or injury. The nucleic acid molecule is delivered to those cells in a form in which it can be taken up by the cells such that sufficient levels of protein can be produced to promote axonal outgrowth or decrease/block an axonal regeneration inhibitor. Alternatively, an expression vector comprising a nucleic acid molecule encoding sialidase is used to produce a transgenic cell, such as a transgenic Schwann cell, and the transgenic cell expressing the sialidase is administered to a site of CNS disease or injury.

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a full length sialidase gene, or a portion thereof, can be cloned into a retroviral vector and expression can be driven from its endogenous promoter, from the retroviral long terminal repeat, or from a promoter specific for a target cell type of interest (e.g., a cell of the central nervous system). Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cometta et al Anderson, Science 226:401-409, 1984; Moen, Blood Cells 17:407-416, 1991; Miller et al., Biotechnology 7:980-990,1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). Most preferably, a viral vector is used to administer the gene of interest systemically or to a cell at the site of a CNS disease or injury.

Non-viral approaches can also be employed for the introduction of therapeutic to a cell of a patient having a CNS disease or injury. For example, a nucleic acid molecule can be introduced into a cell by administering the nucleic acid in the presence of lipofectin (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue at the site of disease or injury. In one embodiment, the transplantation occurs during a peripheral nerve graft to enhance axonal outgrowth from the CNS.

cDNA expression for use in such methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types, such as an intestinal epithelial cell, can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Screening Assays

Compositions having sialidase activity or having sialic acid modifying activity are useful for enhancing axonal outgrowth. Based in part on this discovery, compositions of the invention are useful for the high-throughput low-cost screening of candidate agents, including polypeptides, biologically active fragments, variants, and analogs thereof that have increased activity in axonal outgrowth assays, increased enzymatic activity, enhanced stability, increased enzymatic specificity, reduced toxicity, or an increased ability to cross the blood brain barrier. In one approach, an optimized sialidase polypeptide variant or analog is identified by screening a library of degenerate polypeptides for those that have a desired characteristic, such as enhanced stability or biological activity. There are many ways by which a library of potential bioactive analogs can be generated. In one embodiment, a library of sialidase or chondroitinase variants is generated by combinatorial mutagenesis at the nucleic acid level, and is encoded by a gene library. For instance, a mixture of synthetic oligonucleotides can be enzymatically ligated into gene sequences such that the degenerate set of potential sialidase sequences are expressible as individual polypeptides or as a set of polypeptides. Chemical synthesis of a degenerate gene sequence can be carried out in an automatic DNA synthesizer. The synthetic gene is then ligated into an appropriate expression vector. The purpose of a degenerate set of genes is to provide, in one mixture, all of the sequences encoding the desired bioactive analogs. The synthesis of degenerate oligonucleotides is well known in the art (see for example, Narang, SA (1983) Tetrahedron 39:3; Itakura et al. (1981) Recombinant DNA, Proc 3rd Cleveland Sympos. Macromolecules, ed. A G Walton, Amsterdam: Elsevier pp. 273-289; Itakura et al. (1984) Science 198:1056). Such techniques have also been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382as well as U.S. Pat. Nos. 5,223,409, 5,198,346, and 5,096,815).

Having generated one or more variants of sialidase, various methods may be used to identify variants with the desired properties, i.e., those having enhanced stability, those that block axonal regeneration inhibitor activity, or those having enhanced axonal outgrowth stimulating activity. Whether one or more changes in the amino acid sequence of a peptide results in a bioactive analog can be readily determined by assessing the ability of the variant peptide to produce a response in cells in a fashion similar to the wild-type peptide or competitively inhibit such a response. In addition, the ability of such a polypeptide to biochemically modify a target axonal regeneration inhibitor can also be determined.

A wide range of techniques are known in the art for screening gene products of combinatorial libraries, and for screening cDNA libraries for gene products having a certain property. The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of variant sequences created by combinatorial mutagenesis techniques.

In other embodiments, chemically modified agents having sialidase or sialic acid modifying activity are provided. A polypeptide may be chemically modified to create derivatives by forming conjugates with other chemical moieties, such as glycosyl groups, lipids, phosphate, acetyl groups and the like. Covalent derivatives may be prepared by linking the chemical moieties to functional groups on amino acid side chains or at the N-terminus or at the C-terminus of the polypeptide. For instance, a bioactive agent can be generated which includes a moiety, other than sequences naturally associated with the protein, that binds a component of the extracellular matrix and enhances localization of the analog to cell surfaces.

Polypeptide agents useful in the invention are preferably substantially purified from their source material, be it cell culture, tissue sample, biological fluid, or other biological material. Substantially purified means that the purified material is at least 60% by weight (dry weight) the polypeptide of interest, e.g., a sialidase polypeptide. Preferably, the polypeptide composition is at least 75% or 85%, more preferably at least 90%, and most preferably at least 99%, by weight, the polypeptide of interest. Purity can be measured by any appropriate standard method, e.g., column chromatography, polyacrylamide gel electrophoresis, or HPLC analysis. Substantially purified polypeptides can then be combined with other desired components, such as carriers or cells, to give a composition that is less than 60% composed of polypeptide, so long as the polypeptide is at sufficient concentration to be effective when administered to a patient.

Polypeptide agents useful in the invention can be naturally occurring, synthetic, or recombinant molecules consisting of a hybrid or chimeric polypeptide with one portion, for example, being sialidase and a second portion being a distinct polypeptide. These factors can be purified from a biological sample, chemically synthesized, or produced recombinantly by standard techniques (see.e.g., Ausubel et al., Current Protocols in Molecular Biology, New York, John Wiley and Sons, 1993; Pouwels et al., Cloning Vectors: A Laboratory Manual, 1985, Suppl. 1987).

In one embodiment, sialidase activity is evaluated immunohistochemically as described in Example 1, where enzyme efficacy was evaluated immunohistochemically by assaying the loss GT1b and the gain of GM1 immunostaining. In another embodiment, the sialidase activity of a candidate agent is assayed in an in vitro assay using 2′-(4-methylumbelliferyl)-α-D-N-acetylneuraminic acid (Sigma) as a substrate as described by Hara et al., (Anal Biochem. 1987;164:138-145). Briefly, the fluorogenic substrate is added to the candidate agent under conditions suitable for enzyme activity. Fluorescence measurement is then used to indicate the presence or absence of sialidase activity. Chondroitinase ABC activity is assayed as described in Example 1, where chondroitinase ABC cleaved CSPG chains as evidenced by the gain of immunostaining of the lyase product.

Alternatively or subsequent to such enzymatic assays, agents are assayed in vitro or in vivo for their ability to block axonal regeneration inhibitor activity or to enhance axonal outgrowth. Tissues or cells treated with a candidate agent are compared to untreated control samples to identify therapeutic agents that enhance axonal outgrowth. In vivo assays for the effect of an agent on axon outgrowth are described herein in Example 2. In vitro assays for axonal outgrowth are known in the art. For example, assays for axon outgrowth from rat cerebellar granule neurons in vitro is described by Vyas et al., (J. Biol. Chem. 280:16305-16310, 2005), assays for axon outgrowth from dorsal root ganglion neurons in vitro is described by DeBellard et al., (Mol. Cell. Neurosci. 7: 89-101, 1996), and Assays for axon outgrowth from stem-cell derived motoneurons is described by Harper et al, (Proc. Natl. Acad. Sci. USA 101:7123-7128). Any number of methods are available for carrying out screening assays to identify candidate agents having sialidase activity, having sialic acid modifying activity, or having axonal outgrowth enhancing activity.

In one working example, candidate agents are added at varying concentrations to the culture medium of neuronal cells plated on a composition comprising an axonal regeneration inhibitor. Axonal outgrowth is then measured using standard methods. The level of outgrowth in the presence of the candidate agent is compared to the level measured in a control culture lacking the candidate agent. An agent that promotes axonal outgrowth or that decreases or reverses axonal regeneration inhibitor activity is considered useful in the invention; such an agent may be used, for example, as a therapeutic to prevent, delay, ameliorate, stabilize, or treat a CNS injury or disorder (e.g., spinal cord injury, such as brachial plexus avulsion). In other embodiments, the agent prevents, delays, ameliorates, stabilizes, or treats a disease or disorder characterized by a need for axonal outgrowth or an excess of axonal regeneration inhibitor activity. Such therapeutic compounds are useful in vivo for the promotion of axonal outgrowth following restorative surgery.

In yet another working example, candidate agents are screened for those that specifically bind to an axonal regeneration inhibitor. The efficacy of such a candidate compound is dependent upon its ability to interact with the axonal regeneration inhibitor, or with functional equivalents thereof. Such an interaction can be readily assayed using any number of standard binding techniques and functional assays (e.g., those described in Ausubel et al., supra). In one embodiment, the agent is assayed in a cell in vitro for binding and for the promotion of axonal outgrowth. In one particular working example, a candidate agent that binds to an axonal regeneration inhibitor is identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for an axonal regeneration inhibitor is identified on the basis of its ability to bind to the polypeptide and be immobilized on the column. To isolate the compound, the column is washed to remove non-specifically bound molecules, and the compound of interest is then released from the column and collected. Similar methods may be used to isolate a compound bound to a polypeptide microarray. Compounds and polypeptides identified using such methods are then assayed for their ability to decrease or block an axonal regeneration inhibitor or to enhance axonal outgrowth as described herein.

Agents identified by these methods (or any other appropriate method) may, if desired, be further purified (e.g., by high performance liquid chromatography). Agents isolated by this approach may be used, for example, as therapeutics to treat CNS injury or disease in a subject.

In another embodiment, a nucleic acid encoding a polypeptide having sialidase or sialic acid modifying activity is expressed in an isolated cell (e.g., bacterial, mammalian or insect cell) under the control of an endogenous or a heterologous promoter. The heterologous enzyme, biologically active fragment, or analog thereof is then isolated and tested for activity in an in vitro assays for sialidase activity, for its ability to decrease or block an axonal regeneration inhibitor, or for its ability to promote axonal outgrowth.

Selected candidate agents are then tested in an in vivo model of axonal outgrowth, such as the rat brachial plexus avulsion model, a spinal cord contusion model, or a spinal cord lesion model. One skilled in the art appreciates that the effects of a candidate agent on axonal outgrowth is typically compared to axonal outgrowth in the absence of the candidate agent. Thus, the screening methods include comparing the value of a cell modulated by a candidate agent to a reference value of an untreated control cell.

Sialidase expression or activity can be compared by procedures well known in the art for evaluating enzyme activity. Methods for evaluating enzyme expression or activity include Western blotting, flow cytometry, immunocytochemistry, binding to magnetic and/or antibody- coated beads, in situ hybridization, fluorescence in situ hybridization (FISH), flow chamber adhesion assay, and ELISA, microarray analysis, RT-PCR, Northern blotting, or colorimetric assays, such as the Bradford Assay and Lowry Assay.

In vivo changes in axonal outgrowth or connectivity can also be assayed in functional assays for neurological activity or by neural imaging studies using any method known in the art, including magnetic resonance imaging, PET, axon outgrowth can be determined by retrograde or antegrade labeling in vivo. Typically, a biotin-labeled or florescent dye is injected into the nervous system where it is taken up by neurons and their axons. This allows axons to be traced following fixation and appropriate staining. Functional connectivity can be determined using physiologic tests. These may include, for example, changes in blood pressure responses after renal nerve stimulation. Alternatively, functional connectivity can be determined by testing motor behavior. This may include measuring the time a test animal can remain balanced on a rotating drum or observation of open field walking patterns

Each of the DNA sequences encoding polypeptides listed herein (i.e., sequences encoding a sialidase or chondroitinase) may also be used in the discovery and development of a therapeutic compound for the treatment of a CNS injury or disease. The encoded protein, upon expression, can be used as a target for the screening of drugs that enhance its activity. Additionally, the DNA sequences encoding the amino terminal regions of the encoded protein or Shine-Delgarno or other translation facilitating sequences of the respective mRNA can be used to construct sequences that promote the expression of the coding sequence of interest. Such sequences may be isolated by standard techniques (Ausubel et al., supra).

Test Compounds and Extracts

In general, agents having sialidase activity, sialic acid modifying activity, agents that block axonal regeneration inhibitor activity, or agents that enhance axonal outgrowth are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Agents used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to have sialidase activity, chondroitinase activity, to block axonal regeneration inhibitor activity, or to enhance axonal outgrowth, further fractionation of the positive lead extract is necessary to isolate chemical constituents responsible for the observed effect. Thus, the goal of the extraction, fractionation, and purification process is the careful characterization and identification of a chemical entity within the crude extract that blocks axonal regeneration inhibitor activity, that enhances axonal outgrowth, or that has sialidase or chondroitinase activity. Methods of fractionation and purification of such heterogenous extracts are known in the art. If desired, compounds shown to be useful as therapeutics for the treatment of CNS disease or injury are chemically modified according to methods known in the art.

Pharmaceutical Therapeutics

The invention provides a simple means for identifying compositions (including nucleic acids, peptides, small molecule inhibitors, and mimetics) capable of acting as therapeutics for the treatment of CNS injury or trauma. Accordingly, a chemical entity discovered to have medicinal value using the methods described herein is useful as a drug or as information for structural modification of existing compounds, e.g., by rational drug design. Such methods are useful for screening compounds having an effect on a variety of neurological conditions where an increase in axonal outgrowth is required.

For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a sialidase therapeutic in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the CNS injury. A compound is administered at a dosage that decreases the level or activity of an axonal regeneration inhibitor or that increases axonal outgrowth and the establishment or restoration of sensorimotor function as determined by neurological assays known to the skilled artisan.

Formulation of Pharmaceutical Compositions The administration of a compound for the treatment of a CNS injury or for restoration of neurological function may be by any suitable means that results in a concentration of the therapeutic that, combined with other components, is effective in ameliorating, reducing, or stabilizing a neurological deficit or disorder, such as a spinal cord injury. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in the central nervous system or cerebrospinal fluid; (v) formulations that allow for convenient dosing, such that doses are administered, for example, once every one or two weeks; and (vi) formulations that target the site of a CNS injury where axonal outgrowth is inhibited by inflammation or the presence of residual myelin by using carriers or chemical derivatives to deliver the therapeutic agent to a particular cell type (e.g., neuronal cell) whose function is perturbed by CNS trauma or disease. For some applications, controlled release formulations obviate the need for frequent dosing to sustain the enzyme activity at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active sialidase therapeutic(s), the composition may include suitable parenterally acceptable carriers and/or excipients.

As indicated above, the pharmaceutical compositions according to the invention may be in the form suitable for sterile injection. To prepare such a composition, the suitable active active sialidase therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate).

The active sialidase therapeutic(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents. Alternatively, the active drug may be incorporated in biocompatible carriers, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

Formulations for oral use include tablets containing the active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Methods of Delivery

The pharmaceutical compositions of this invention comprising agents having sialidase or sialic acid modifying activity, including sialidase polypeptides, biologically active fragments, variants, or analogs thereof, can be administered by any suitable routes including intracranial, intracerebral, intraventricular, intrathecal, intraspinal, oral, topical, rectal, transdermal, subcutaneous, intravenous, intramuscular, intranasal, and the like. In one embodiment, the compositions are added to a retained physiological fluid, such as cerebrospinal fluid, blood, or synovial fluid. The disclosed therapeutic agents are amenable to direct injection or infusion at a site of CNS disease or injury. In one approach, a therapeutic of the invention is provided within an implant, such as an osmotic pump, or in a graft comprising appropriately transformed cells (i.e., cells expressing sialidase or chondroitinase). Methods of introduction may also be provided by rechargeable or biodegradable devices. Various slow release polymeric devices have been developed and tested for the controlled delivery of drugs, including proteinacious biopharmaceuticals. A variety of biocompatible polymers (including hydrogels), including both biodegradable and non-degradable polymers, can be used to form an implant for the sustained release of a bioactive factor at a particular target site. Generally, the amount of administered agent having sialidase or chondroitinase ABC activity will be empirically determined. Typically agents are administered in the range of about 10 to 1000 μg/kg of the recipient. For peptide agents, the concentration will generally be in the range of about 50 to 500 μg/ml in the dose administered. In other embodiments, therapeutic agent dosages of about 0.1, 0.2, 0.3, 0.38, 0.5, 1, 2, 3, 4, and 5.0 U/ml are used. Other additives may be included, such as stabilizers, bactericides, and anti-fungals. These additives will be present in conventional amounts.

Kits

The invention provides kits for the treatment of CNS disease or injury. In one embodiment, the kit includes a therapeutic composition containing an effective amount of a sialidase or chondroitinase ABC polypeptide or expression vector encoding a therapeutic sialidase or chondroitinase ABC polypeptide in unit dosage form. In some embodiments, the kit comprises a sterile container which contains a therapeutic; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired a therapeutic of the invention is provided together with instructions for administering it to a subject having a CNS disease or injury. The instructions will generally include information about the use of the composition for enhancing axonal outgrowth. In one embodiment, the instructions will include information regarding the use of the therapeutic prior to, during, or after restorative surgery, such as a peripheral nerve graft. In other embodiments, the instructions include at least one of the following: description of the polypeptide or expression vector; dosage schedule and administration for treatment of CNS disease or injury or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

### Example 1

Sialidase and Chondroitinase ABC Exhibit in Vivo Activity

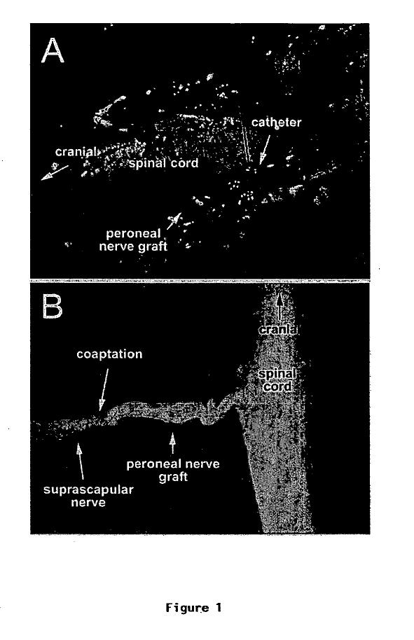

The C8 brachial plexus was lesioned adjacent to the spinal cord in rats, and an autologous peroneal nerve graft was inserted into the spinal cord at the same site (FIGS. 1A and 1B). This model is analogous to human brachial plexus avulsion injury combined with the therapeutic implantation of a peripheral nerve graft into the spinal cord with coaptation of the distal end of the graft to a local (suprascapular) peripheral nerve. Three enzymes that interrupt the actions of different ARI's, PI-PLC, sialidase, and chondroitinase ABC, were delivered to the graft insertion site with a loading dose, then for 14 days via a catheter attached to an osmotic pump. The concentrations of chondroitinase ABC (0.5 and 5.0 U/ml) equal and exceed those effective in prior in vivo studies²¹⁻²³. The concentrations of PI-PLC (2 and 20 U/ml) and sialidase (0.1 and 0.38 U/ml) equal and exceed those effective in vitro^{6,11-12}.

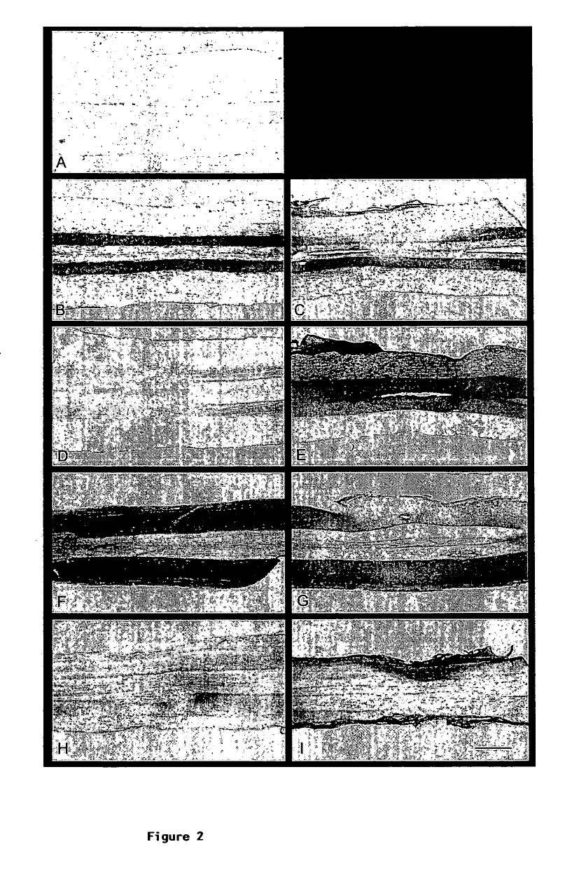

Enzyme efficacy in vivo was confirmed by immunohistochemistry (FIGS. 2A-2I). The efficacy of sialidase, PI-PLC and chondroitinase ABC in vivo were evaluated immunohistochemically after infusion to a thoracic spinal cord lesion under conditions mimicking those used in therapeutic experiments. Sialidase cleaved terminal sialic acids as evidenced by the loss GT1b and the gain of GM1 immunostaining. PI-PLC released GPI-anchored proteins as evidenced by loss of Thy-1 immunostaining, and chondroitinase ABC cleaved CSPG chains as evidenced by the gain of immunostaining of the lyase product. When infused in vivo, the enzymes did not result in overt toxicity based on the behavior of treated animals over the course of the experiment. Histological examination of fixed spinal cords revealed that none of the treatments caused tissue damage except for 5 U/ml chondroitinase ABC, which induced tissue deterioration when administered at 5 U/ml.

### Example 2

Sialidase and Chondroitinase ABC Enhance Axonal Outgrowth in Vivo

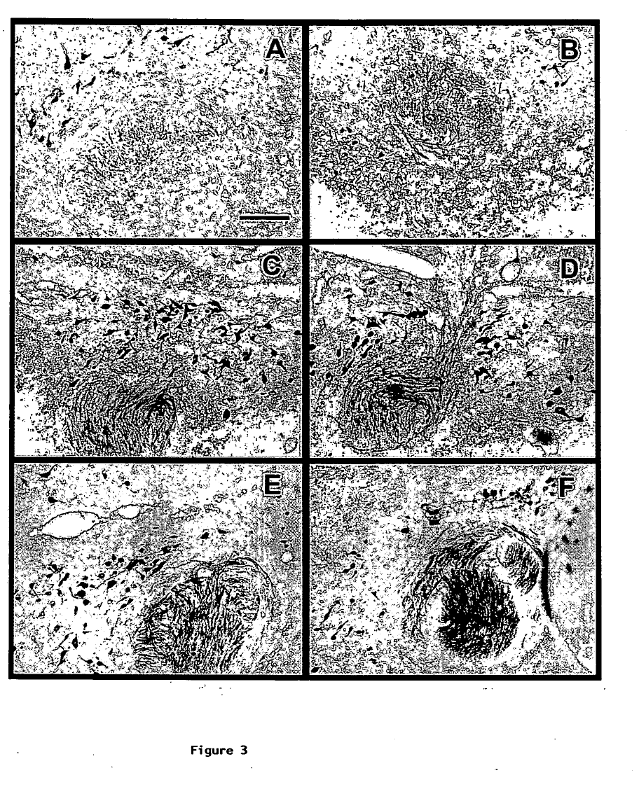

Four weeks after graft implantation, the number of spinal axons extending well into the peroneal nerve graft was determined by retrograde labeling. Uniform, reproducible, and complete retrograde labeling of the peripheral nerve graft was accomplished by transecting the graft 7 mm distal to its insertion into the spinal cord, then immersing and sealing the proximal end into a micro-reservoir filled with Fluoro-Ruby dye. Three days later, rats were sacrificed and the number of retrograde labeled spinal neurons was determined microscopically. Labeled neurons, which had extended axons well into the peripheral nerve graft, were observed in the ventral horn near the site of the implant (FIGS. 3A-3D). In control animals the graft was sutured in place but not inserted into the spinal cord. When these grafts were fitted with micro-reservoirs, no spinal neurons were stained indicating that stained spinal neurons were retrogradely labeled only via graft innervation.

Control (saline-treated) animals displayed some innervation of the graft (FIG. 3A), which is consistent with the clinical use of peripheral nerve grafts for brachial plexus avulsion injury, albeit with limited efficacy. Graft innervation in animals treated with PI-PLC (2 or 20 U/ml) was similar to that of controls (FIG. 3B). Notably, many more spinal neurons were retrogradely labeled in animals treated with sialidase (0.38 U/ml, FIGS. 3C,D) or chondroitinase ABC (0.5 U/ml, FIGS. 3E,F). Quantitative analyses confirmed this conclusion (FIGS. 3A-3F). Analysis of variance (ANOVA) indicated highly significant differences among the test groups (p<0.005). Innervation increased 2.6-fold (p=0.005). Treatment with chondroitinase ABC (0.5 U/ml) resulted in a similar, 2.5-fold increase in innervation (p=0.002). In contrast, treatment with a lower concentration of sialidase (0.1 U/ml) or with PI-PLC (2 or 20 U/ml) resulted in small increases in innervation that were not significantly different from controls (p>0.4).

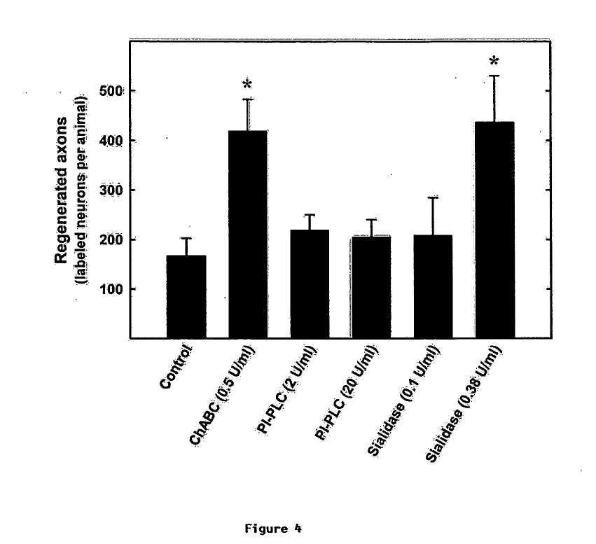

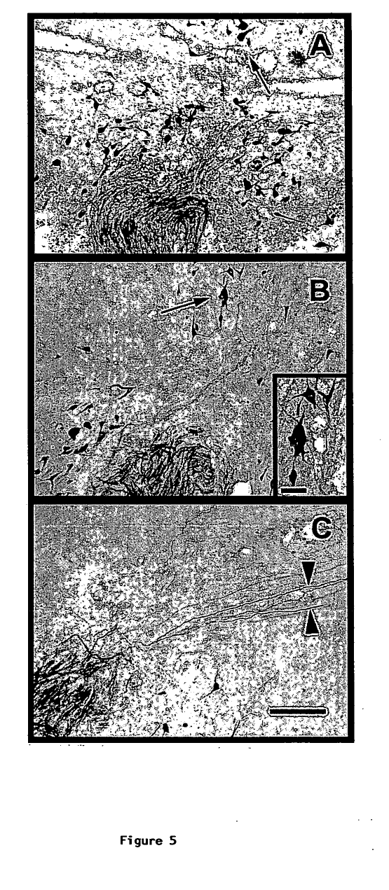

In sialidase-treated (0.38 U/ml) and chondroitinase ABC-treated (0.5 U/ml) animals, most retrogradely labeled neurons were near the graft implant site (FIGS. 3C-F), suggesting that enhanced axon outgrowth was locally stimulated. Labeled neurons were also found in the contralateral ventral horn and adjacent to the central canal (arrows, FIGS. 5A,B). The total number of labeled neurons in all horizontal sections of grafted animals (averaging ˜170 in control and >400 in sialidase- and chondroitinase ABC-treated animals, FIG. 4) was consistent with the intense axonal staining of axons exiting the spinal cord in the initial graft segment, especially in animals receiving effective treatment (FIGS. 3A-3F and 5A-5C). This indicated that most of the labeled axons exiting at the initial graft segment emanated from cell bodies that reside in horizontal sections dorsal and ventral to the insertion site. Examples of the continuity of labeled axons with their corresponding cell bodies within a single horizontal section were also identified (color-highlighted, FIGS. 5A,B), and occasional labeled axons spanned multiple segments cranial or caudal to the site of implantation (FIG. 5C).

Delivery of sialidase or chondroitinase ABC to a brachial plexus injury site enhanced innervation of peripheral nerve grafts >2.5-fold compared to animals that received saline alone or PI-PLC. These are the first data demonstrating a potential therapeutic benefit of sialidase in CNS injury. These findings add to growing evidence that blocking or reducing the activity of axonal regeneration inhibitors can enhance axon regeneration in vivo and promote recovery after CNS injury²⁴.

The mechanism by which sialidase enhances axon outgrowth in vivo has yet to be established. Without wishing to be bound by theory, in one embodiment sialidase destroys axonal receptors for MAG, such as gangliosides GD1a and GT1b^{6,8,9,25,26}. The C. perfringens sialidase used in the current study cleaved the terminal sialic acids from gangliosides at the site of enzyme infusion (see FIGS. 2A-2I). Immunohistochemistry was consistent with the action of each enzyme on its respective substrate(s) in vivo. Sialidase infusion resulted in an asymmetric decrease in GT1b immunostaining (FIGS. 5B,C) and increase in GM1 immunostaining (FIGS. 2D,E) at the site of infusion. Although Thy-1 immunostaining was not uniform at all anatomic levels of control sections, an asymmetric decrease in immunostaining was revealed at the site of infusion of PI-PLC (FIGS. 2F,G). Finally, staining with monoclonal antibody 2-B-6 revealed asymmetric appearance of lyase product in rats infused with chondroitinase ABC (FIGS. 2H,I).

It is, therefore, likely that enhanced axon outgrowth after sialidase treatment was due to destruction of GD1a and GT1b, resulting in loss of MAG-mediated inhibition. Alternatively, C. perfringens sialidase may act on gangliosides, sialoglycoproteins, and polysialic acid²⁷, which might affect axon outgrowth. Furthermore, the product of sialidase action on major brain gangliosides, GM1, may have protective or trophic effects independent of MAG²⁸.

Regardless of which specific sialoglycoconjugates are involved, these results indicate that sialidase is useful for enhancing axon outgrowth in vivo. Moreover, the effect induced by sialidase is likely to be mechanistically distinct from that induced by chondroitinase ABC. The sugar chains of CSPG are unaffected by sialidase, and sialoglycoconjugates are unaffected by chondroitinase ABC. No evidence implicates a functional link between sialoglycans and CSPG's in the inhibition of axon regeneration. This suggests that combination therapy that provides sialidase and chondroitinase ABC will increase the enhancement of axon outgrowth.

Quantitatively, stimulation of axon outgrowth into the peripheral nerve graft by sialidase and chondroitinase ABC was very robust. FIG. 4 indicated an increase in the number of axons extending into the graft from ˜170 (control) to >400 (sialidase and chondroitinase ABC). This level of innervation is comparable to that observed when C7 innervation is quantitated by retrograde labeling in intact animals. Jivan et al.²⁹, for example, reported that 440 motoneurons were labeled when the transected proximal end of the ventral branch of the C7 spinal nerve was immersed in Fast Blue, a very efficient retrograde label. In the same report, retrograde labeling through a peripheral nerve graft (16 weeks post grafting) resulted in only 170 labeled neurons, which is consistent with results in untreated animals reported herein.

Spinal cord neurons induced to extend axons into peripheral nerve grafts when treated with sialidase and chondroitinase ABC were not strictly limited to those in the ventral horn adjacent to the graft insertion (FIG. 5). This finding is in agreement with prior studies on the effects of an acute implantation of an avulsed lumbar ventral root into the rat spinal cord, in which a few contralateral neurons reinnervated the implant³⁰. Likewise, neurons from multiple spinal cord segments have been shown to extend axons into a single re-implanted ventral root³¹ . The morphology of the retrogradely labeled neurons in the current study was consistent with their identification, predominantly, as motoneurons.

The results reported herein establish for the first time that sialidase enhanced axon outgrowth in an in vivo animal model. It is likely that sialidase, alone or in combination with other axonal regeneration inhibitor blockers, will enhance axon outgrowth in other nerve injury models. The finding that chondroitinase ABC independently enhanced axon outgrowth in the same model suggests that targeting axonal regeneration inhibitors, especially sialoglycans and CSPG's, will enhance axonal outgrowth and functional recovery following restorative surgical treatment for brachial plexus avulsion injuries.

The preceding results were obtained using the following methods and materials.

Enzymes and Osmotic Pumps

The following enzymes were diluted in sterile Dulbecco's phosphate buffered saline without calcium or magnesium as follows: PI-PLC from Bacillus cereus, 2 or 20 U/ml (P-5542, Sigma-Aldrich, St. Louis, Mo.), sialidase from Clostridium perfringens, 0.1 or 0.38 U/ml (neuraminidase, 480708, Calbiochem, San Diego, Calif.), and chondroitinase ABC from Proteus vulgaris, 0.5 or 5.0 U/ml (Seikagaku 100332, Associates of Cape Cod, East Falmouth, Mass.). Osmotic pumps (Alzet, 200 μl, 0.5 μl/hr, 14 days; Durect Corp, Cupertino, Calif.) were equipped with 5 cm of PE-60 tubing secured with 2-0 silk, filled with sterile saline or enzyme solution, then pre-incubated in sterile saline at 37 ° C. overnight to prime the pump.

Peripheral Nerve (Graft) Harvest.

Male Sprague-Dawley rats weighing 200-250 g were subjected to continuous halothane anesthesia after induction. After sterilizing the skin, an incision was made over the line of the peroneal nerve and taken down to reveal the nerve, which was sharply dissected free of the surrounding tissues from the ankle to the takeoff from the sciatic nerve. A 2-3 cm segment was harvested and placed in sterile gauze moistened with saline until use.

Preparation of the Graft Site.

After sterilizing the skin, a midline posterior cervical incision was made to expose the cervical musculature. The lateral edge of the trapezius and the attachment of the trapezius to the spine of the scapula was also exposed. The attachment of the upper trapezius to the scapular spine was detached, revealing the attachment of the omohyoid to the region of the suprascapular notch. The suprascapular nerve approaching the suprascapular notch was visualized anterior to the supraspinatus muscle, dissected free of the surrounding connective tissues and transected 1 cm anterior to the suprascapular notch.

Nerve Root Avulsion, and Graft Implantation

After cervical laminectomy (C6-T1), the paraspinous muscles were transected in the line of graft implantation. The distal end of the graft was coapted to the recipient suprascapular nerve with 9-0 nylon suture. The PE-60 tubing from the osmotic pump was then tunneled parallel to the spine under 2 cm of paraspinous musculature and cut so that the end lay immediately caudal to the intended site of graft insertion. The tubing was anchored to the edge of the dura and adjacent muscle with 8-0 nylon so that the opening of the tubing lay intradural adjacent to the left side of the spinal cord. To model nerve avulsion, C8 dorsal and ventral rootlets were transected at the transitional zone. The proximal end of the peroneal nerve graft was then implanted 1.5 mm into the ventrolateral aspect of the C8 spinal cord using a fine beveled syringe tip. The epineurium of the graft was secured to the dura with 9-0 nylon. Immediately after graft insertion, 50 μl of saline or enzyme solution (the same solution used to load the osmotic pump), was introduced intradurally to the operative site. The trapezius and paraspinous muscles were reapproximated with 4-0 silk suture and the skin closed with surgical staples.

Retrograde Labeling with Fluoro-Ruby

Four weeks after the initial operation, under continuous halothane anesthesia, rats underwent a second operation for retrograde labeling via the peripheral nerve graft. A 2-cm incision lateral to the prior cervical incision was made and taken sharply through the subcutaneous tissues. The sutures reapproximating the trapezius were cut to reveal the suprascapular nerve and its coaptation to the graft. The graft was traced medially until the suture securing the epineurium of the graft to the dura was visualized (marking the lateral edge of the spinal canal). The graft was transected 7 mm lateral (distal) to the suture, and the newly cut end was inserted into a micro-reservoir consisting of the 3-mm tip of a heat-sealed 200 μl micropipet tip containing 5 μl of 5% Fluoro-Ruby dye (tetramethylrhodamine/lysine dextran, D1817, Invitrogen-Molecular Probes, Carlsbad, Calif.) in sterile water. Approximately 100 μl of Tisseel fibrin sealant (Baxter, Deerfield, Ill.) was added to seal the top of the reservoir and ensure continuity of the cut distal end of the graft with the enclosed retrograde tracer. The trapezius and paraspinous muscles were reapproximated with 4-0 silk suture and the skin closed with surgical staples.

Perfusion Fixation, Sectioning, and Microscopy

Three days after initiating retrograde labeling, under halothane anesthesia, saturated urethane in water (2 ml) was injected intraperitoneally. When the rat lacked spontaneous breathing, transcardial perfusion was initiated with saline and then 4% paraformaldehyde in saline. The spinal cord in continuity with the peripheral nerve graft was removed en bloc (see Fig. 1B) and post-fixed in 4% paraformaldehyde overnight, followed by cryoprotection in 30% sucrose for 24 h. Horizontal sections (40 μm) were cut using a freezing microtome and mounted on glass slides. Fluorescent images were obtained with a SONY CCD camera attached to a NIKON TE200 fluorescence microscope using rhodamine filters. Retrogradely labeled neurons in all sections were counted; to reduce the likelihood of double-counting, only neurons in which the nucleus was apparent were counted. Counting was performed by investigators blind to the treatment group. Statistical comparisons among groups was by ANOVA, and between each enzyme-treated group and the saline control group by Student's T test.

Antibodies and Immunochemicals for Test of Enzyme efficacy in Vivo

Anti-ganglioside monoclonal antibodies were prepared as described previously (Schnaar et al. (2002) Anal. Biochem. 302, 276-284). Anti-GT1b (GT1b-2b) and anti-GM1 (GM1-1) were used at final concentrations of 0.5 and 1.1 μg/ml respectively. C. perfringens sialidase converts GT1b and other abundant complex gangliosides to GM1, but fails to cleave the single sialic acid from GM1 (Schauer et al., (1980) Adv. Exp. Med. Biol. 125, 283-294). Therefore, sialidase efficacy was revealed by a decrease in GTIb immunostaining and a concomitant increase GM1 immunostaining. PI-PLC efficacy was revealed by the decrease in immunostaining for an abundant nervous system GPI-anchored protein, Thy-1, using anti-Thy-1 mouse monoclonal antibody (0.5 μg/ml final concentration) from Chemicon International, Temecula, Calif. (product CBL1500). Chondroitinase ABC efficacy was revealed by an increase in immunostaining with mouse monoclonal antibody 2-B-6 (10 μg/ml final concentration, Associates of Cape Cod, East Falmouth, Mass.), which binds to the lyase product of chondroitin sulfate cleavage (Sorrell et al., (1988) J. Immunol. 140, 4263-4270). Primary antibody binding was detected using biotin-conjugated goat anti-mouse IgG (Fc specific, Jackson ImmunoResarch, West Grove, Pa., USA), avidin/biotinylated alkaline phosphatase conjugate (Vector Laboratories, Burlingame, Calif., USA), and Vector Red alkaline phosphatase substrate (Vector Laboratories).

Surgery and Enzyme Infusion for Test of Enzyme Efficacy in vivo

Under halothane anesthesia, rats (Charles River, ˜250 g) were subjected to a ventral midline incision to expose the thoracic musculature and the paraspinous muscles removed from dorsal spines T8-T10and T12-T13. A laminectomy was performed at T9 and a partial laminectomy at the T12/T13 junction. PE-60 tubing, pulled to a diameter of ˜200 μm, was inserted through a small incision in the dura at T12/T13, fed rostrally until the tip lay just caudal to T9, and the catheter sutured to muscle. A small incision was made in the dura and a small lesion was made to the lateral spinal cord at T9 by compressing with a fine forceps (to mimic peripheral nerve implantation). Immediately after compression, 50 μl of enzyme solution (C. perfringens sialidase, 0.4 U/ml; PI-PLC, 2 U/ml; or chondroitinase ABC, 0.5 U/ml) was introduced through the catheter to the lesion site and the proximal end of the catheter was connected to a subcutaneous osmotic pump containing the same enzyme solution (control animals received the spinal cord lesion but no enzyme). The incision was closed in two layers.

Five days after the initial surgery, rats were anesthetized, sacrificed and perfused with 4% paraformaldehyde. The thoracic spinal cord was exposed and the rostrocaudal position of the catheter tip marked by insertion of a pin, from the dorsal to the ventral surface, into the fixed spinal cord. The spinal cord was dissected, postfixed, cryoprotected and 40 μm horizontal sections prepared using a freezing microtome as described in the text.

Immunohistochemistry for Test of Enzyme Efficacy in vivo

Free-floating spinal cord sections were blocked for 2 hours at ambient temperature in IHC buffer (Tris-buffered saline containing 10 mg/ml of bovine serum albumin and 5% (v/v) goat serum), then incubated overnight at 4° C. in primary antibody in the same buffer. Sections were washed three times in Tris-buffered saline and incubated 4 hours at 4° C. in biotin-conjugated goat anti-mouse IgG (2 itg/ml in IHC buffer). Sections were washed as described above and incubated with avidin/biotinylated alkaline phosphatase conjugate (Vector Laboratories, Burlingame, Calif., USA) for 2 hours, and developed with Vector Red alkaline phosphatase substrate (Vector Laboratories) according to the manufacturer's instructions. Stained and washed sections were transferred to glass slides, dried, mounted and images collected under brightfield illumination. In enzyme-treated rats, the rostrocaudal position of the tip of the infusion catheter was revealed by a pin hole appearing at the same relative position in adjacent horizontal sections.

Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All references, including patents and publications, mentioned in this specification are herein incorporated by reference to the same extent as if each independent patent and publication was specifically and individually indicated to be incorporated by reference.

### REFERENCES

1. Griffiths, I. R. (1974) J. Small Anim Pract. 15, 165-176.
2. He, Z. & Koprivica, V. (2004) Annu. Rev. Neurosci. 27, 341-368.
3. McGee, A. W. & Strittmatter, S. M. (2003) Trends Neurosci. 26, 193-198.
4. Venkatesh, K., Chivatakarn, O., Lee, H., Joshi, P. S., Kantor, D. B., Newman, B. A., Mage, R., Rader, C., & Giger, R. J. (2005) J. Neurosci. 25, 808-822.
5. Varki, A. & Angata, T. (2006) Glycobiology 16, 1R-27R.
6. Vyas, A. A., Patel, H. V., Fromholt, S. E., Heffer-Lauc, M., Vyas, K. A., Dang, J., Schachner, M., & Schnaar, R. L. (2002) Proc. Natl. Acad. Sci. U.S.A 99, 8412-8417.
7. Vyas, A. A., Blixt, O., Paulson, J. C., & Schnaar, R. L. (2005) J. Biol. Chem. 280, 16305-16310.
8. Vinson, M., Strijbos, P. J., Rowles, A., Facci, L., Moore, S. E., Simmons, D. L., & Walsh, F. S. (2001) J. Biol. Chem. 276, 20280-20285.
9. DeBellard, M.-E., Tang, S., Mukhopadhyay, G., Shen, Y.-J., & Filbin, M. T. (1996) Mol. Cell. Neurosci. 7, 89-101.
10. Carulli, D., Laabs, T., Geller, H. M., & Fawcett, J. W. (2005) Curr. Opin. Neurobiol. 15, 116-120.
11. Liu, B. P., Fournier, A., GrandPre, T., & Strittmatter, S. M. (2002) Science 297, 1190-1193.
12. Wang, K. C., Koprivica, V., Kim, J. A., Sivasankaran, R., Guo, Y., Neve, R. L., & He, Z. (2002) Nature 417, 941-944.
13. Collins, B. E., Yang, L. J. S., Mukhopadhyay, G., Filbin, M. T., Kiso, M., Hasegawa, A., & Schnaar, R. L. (1997) J. Biol. Chem. 272, 1248-1255.
14. Narakas, A. 0. (1993) Clin. Neurol. Neurosurg. 95 Suppl, S56-S64.
15. Bertelli, J. A. & Ghizoni, M. F. (2003) Neurosurgery 52, 1385-1389.
16. Holtzer, C. A., Marani, E., Lakke, E. A., & Thomeer, R. T. (2002) J. Peripher. Nerv.

Syst. 7, 233-242.
17. Cullheim, S., Carlstedt, T., & Risling, M. (1999) Spinal Cord. 37, 811-819.
18. Carlstedt, T. (1993) Clin. Neurol. Neurosurg. 95 Suppl, S109-S111 .
19. Carlstedt, T., Grane, P., Hallin, R. G., & Noren, G. (1995) Lancet 346, 1323-1325.
20. Carlstedt, T., Anand, P., Hallin, R., Misra, P. V., Noren, G., & Seferlis, T. (2000) J. Neurosurg. 93, 237-247.
21. Moon, L. D., Asher, R. A., Rhodes, K. E., & Fawcett, J. W. (2001) Nat. Neurosci. 4, 465-466.
22. Bradbury, E. J., Moon, L. D., Popat, R. J., King, V. R., Bennett, G. S., Patel, P. N., Fawcett, J. W., & McMahon, S. B. (2002) Nature 416, 636-640.
23. Caggiano, A. O., Zimber, M. P., Ganguly, A., Blight, A. R., & Gruskin, E. A. (2005) J. Neurotrauma 22, 226-239.
24. David, S. & Lacroix, S. (2003) Annu. Rev. Neurosci. 26, 411-440.
25. Yang, L. J. S., Zeller, C. B., Shaper, N. L., Kiso, M., Hasegawa, A., Shapiro, R. E., & Schnaar, R. L. (1996) Proc. Natl. Acad. Sci. U. S. A. 93, 814-818.
26. Collins, B. E., Kiso, M., Hasegawa, A., Tropak, M. B., Roder, J. C., Crocker, P. R., & Schnaar, R. L. (1997) J. Biol. Chem. 272, 16889-16895.
27. Cassidy, J. T., Jourdian, G. W., & Roseman, S. (1965) J. Biol. Chem. 240, 3501-3506.
28. Wu, G., Lu, Z. H., Wang, J., Wang, Y., Xie, X., Meyenhofer, M. F., & Ledeen, R. W. (2005) J Neurosci. 25, 11014-11022.
29. Jivan, S., Novikova, L. N., Wiberg, M., & Novikov, L. N. (2006) Exp. Brain Res. 170, 245-254.
30. Carlstedt, T., Linda, H., Cullheim, S., & Risling, M. (1986) Acta Physiol Scand. 128, 645-646.
31. Hoang, T. X. & Havton, L. A. (2006) Exp. Brain Res. 169, 208-217.

1. A method for enhancing axonal outgrowth in a cell by contacting the cell or cell substrate with an agent having sialidase or sialic acid modifying activity thereby enhancing axonal outgrowth.

2. The method of claim 1, wherein the agent is sialidase.

3. The method of claim 1, wherein the agent having sialidase activity modifies sialoglycoconjugates or cleaves terminal sialic acids present on the cell or on the cell substrate.

4. The method of claim 1, wherein the agent modifies gangliosides, sialoglycoproteins, or polysialic acid present on the cell or the cell substrate.

5. The method of claim 4, wherein the modified gangliosides are GD1a and GT1b.

6. The method of claim 2, wherein the agent having sialic acid modifying activity modifies a sialic acid present on the cell or in the cellular environment.

7. The method of claim 1, wherein the cell is a cell of the central nervous system.

8. The method of claim 7, wherein the cell is a neuron.

9. The method of claim 8, wherein the cell is a motor or sensory neuron.

10. The method of claim 8, wherein an axon of the cell is present in a brachial plexus.

11. The method of claim 1, wherein the method enhances outgrowth from the CNS into a peripheral nerve graft.

12. The method of claim 1, wherein the agent is administered prior to, during, or following restorative CNS surgery.

13. The method of claim 1, wherein the agent having sialidase activity is administered in combination with an agent having chondroitinase ABC activity.

14. The method of claim 13, wherein the agent having sialidase activity is administered concurrently or within 5 days of administration of the agent having chondroitinase ABC activity.

15. The method of claim 13, wherein between about 0.1 U/ml and 5 U/mi of chondroitinase ABC is administered.

16. The method of claim 1, wherein between about 0.1 and 5 U/mI of sialidase is administered.

17. The method of claim 1, wherein the administration increases axonal outgrowth by at least 2-fold relative to an untreated control condition.

18. A method of blocking an axonal regeneration inhibitor in a subject in need thereof, the method comprising administering to the subject an effective amount of an agent having sialidase or sialic acid modifying activity, thereby blocking an axonal regeneration inhibitor in the subject.

19. A method of enhancing axonal outgrowth in a subject in need thereof, the method comprising administering to the subject an effective amount of an agent having sialidase or sialic acid modifying activity thereby enhancing axonal outgrowth.

20. The method of claim 18, wherein the agent is sialidase.

21-34. (canceled)

35. A method of enhancing axonal outgrowth in a subject having a spinal injury, the method comprising administering to a subject having CNS restorative surgery an effective amount of an agent having sialidase or sialic acid modifying activity thereby enhancing axonal outgrowth.

36-38. (canceled)

39. A method for identifying an agent that enhances axonal outgrowth, the method comprising:
(a) contacting a neuron in the presence of an axonal regeneration inhibitor with an agent having sialidase activity or sialic acid modifying activity; and
(b) comparing axonal outgrowth in the presence of the agent relative to a control condition, wherein an increase in axonal outgrowth in the presence of the agent thereby identifies the agent as enhancing axonal outgrowth.

40. A method for identifying an agent that enhances axonal outgrowth, the method comprising:
(a) contacting an axonal regeneration inhibitor with an agent having sialidase activity or sialic acid modifying activity; and
(b) identifying a biochemical modification of the axonal regeneration inhibitor, wherein an agent that biochemically modifies the axonal regeneration inhibitor is identified as enhancing axonal outgrowth.

41-42. (canceled)

43. A pharmaceutical composition for use in enhancing axonal outgrowth in a subject in need thereof, the composition comprising an effective amount of an agent having sialidase activity or sialic acid modifying activity in a pharmaceutically acceptable excipient.

44-45. (canceled)

46. A therapeutic delivery device comprising an agent having sialidase activity or sialic acid modifying activity, wherein the device locally releases the agent into the CNS for the treatment of a CNS disease or injury.

47-48. (canceled)
